(19) United States
(12) Patent Application Publication
(10) Pub. No.: US 2009/0036876 A1
(43) Pub. Date: Feb. 5, 2009

(54) Gel Cap Loading Tool

(75) Inventor: An Quoc Tran, Ventura, CA (US)

Correspondence Address: FREUDENBERG-NOK GENERAL PARTNERSHIP;LEGAL DEPARTMENT, 47690 EAST ANCHOR COURT, PLYMOUTH, MI, 48170-2455, US

(73) Assignee: HELIX MEDICAL, LLC, Plymouth, MI (US)

(21) Appl. No.: 11/832,167

(22) Filed: Aug. 1, 2007

(51) Int. Cl. A61B 17/00

(52) U.S. Cl. 606/1

(57) ABSTRACT

A medical device, gel-cap loading tool. The loading tool facilitates the insertion of a component of a medical device, such as the esophageal flange of a voice prosthesis, within a dissolvable gel cap. The gel cap is disposed in a cavity with the component collapsed therein. A gel cap is disposed in the cavity and is axially aligned with the medical device. An engaging member engages with at least one of the gel cap and the medical device to cause relative movement between the gel cap and the medical device which causes the component to be inserted into the gel cap.

### FIELD

The present disclosure relates to loading tools and, more particularly, to an apparatus for loading a gel cap over a component of a medical device, such as a flange of a voice prosthesis, wherein a reduction in radial profile is used to fit the medical device into an orifice.

### BACKGROUND AND SUMMARY

The statements in this section merely provide background and summary information related to the present disclosure and may not constitute prior art.

Various medical devices can have a radial profile that is temporarily reduced to fit the medical device into an orifice. Once such medical device is a voice prosthesis. A voice prosthesis is a one-way valve that can be placed into an orifice (puncture) between the trachea and esophagus. The voice prosthesis allows air to flow from the lungs into the esophagus to enable speech. Flanges at both ends of the voice prosthesis retain the prosthesis in the tracheoesophageal puncture.

The insertion of the esophageal flange of the voice prosthesis through the tracheoesophageal puncture can be a traumatic experience. To reduce the traumatic experience, the esophageal flange can be placed within a dissolvable capsule (referred to herein as “gel cap”, “cap” and “capsule”) to reduce the overall dimensions and facilitate the insertion through the tracheoesophageal puncture. Once in place, water or other liquid can be swallowed to dissolve the capsule and allow the esophageal flange to expand to its normal dimension and secure the voice prosthesis within the tracheoesophageal puncture.

Existing devices to load the esophageal flange within the cap may be difficult to use. New and existing users often struggle to properly load the esophageal flange into the cap. The existing devices can have a long learning curve. Additionally, the user typically wears protective gloves when handling the voice prosthesis and, therefore, limits the tactile sensation.

The present disclosure provides a loading tool. The loading tool facilitates the insertion of a component of a medical device, such as the esophageal flange of a voice prosthesis, within a dissolvable gel cap. The gel cap is disposed in a cavity with the component collapsed therein. A gel cap disposed in the cavity can be axially aligned with the medical device. An engaging member engages with at least one of the gel cap and the medical device to cause relative movement between the gel cap and the medical device which causes the component to be inserted into the gel cap.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of the various components of a loading tool according to the present disclosure in an unassembled state along with a voice prosthesis and gel cap;

FIG. 2 is a perspective view of the loading tool of FIG. 1 with the sleeve disposed in the barrel;

FIG. 3 is a perspective view of the loading tool of FIG. 2 with a voice prosthesis inserted into the loading tool;

FIG. 4 is a perspective view of the loading tool of FIG. 3 with a gel cap inserted into the loading tool;

FIG. 5 is a perspective view of the loading tool of FIG. 4 with the inserter stick engaged with the loading tool and the voice prosthesis;

FIGS. 6-7 are perspective views of the loading tool of FIG. 5 in various stages of loading the gel cap over the esophageal flange;

FIG. 8 is a perspective view of the loading tool of FIG. 7 with the gel cap loaded over the esophageal flange and the voice prosthesis coupled to the inserter stick;

FIG. 9 is a perspective view of a voice prosthesis with a gel cap over the esophageal flange;

FIG. 10 is a partial cutaway view of the tracheoesophageal puncture between the trachea and the esophagus with the gel cap dissolved and the esophageal and tracheoesophageal flanges maintaining the voice prosthesis therein;

FIG. 11 is an end view of the sleeve of the loading tool of FIG. 1;

FIG. 12 is a perspective view of an alternate configuration of the loading tool of FIG. 1 according to the present disclosure;

FIG. 13 is an assembled perspective view of the loading tool of FIG. 12 with a voice prosthesis, gel cap, and inserter stick engaged with the loading tool;

FIG. 14-15 are perspective views of the loading tool of FIG. 13 in various stages of loading the gel cap over the esophageal flange;

FIG. 16 is an alternate configuration for a sleeve that can be used with the loading tool of FIG. 1;

FIG. 17 is a perspective view of another loading tool according to the present disclosure in an unassembled state along with a voice prosthesis and gel cap;

FIG. 18 is a perspective view of the loading tool of FIG. 14 in a partially assembled state;

FIG. 19 is a perspective view of the loading tool of FIG. 15 in another partially assembled state;

FIGS. 20-22 are perspective views of the loading tool of FIG. 16 in various stages of loading the gel cap over the esophageal flange; and

FIG. 23 is a perspective view of the loading tool of FIG. 19 with the gel cap on the esophageal flange and the voice prosthesis coupled to the inserter stick and ready to be inserted into the tracheoesophageal puncture.

### DETAILED DESCRIPTION

The following description is merely exemplary in nature and is not intended to limit the present disclosure, application, or uses.

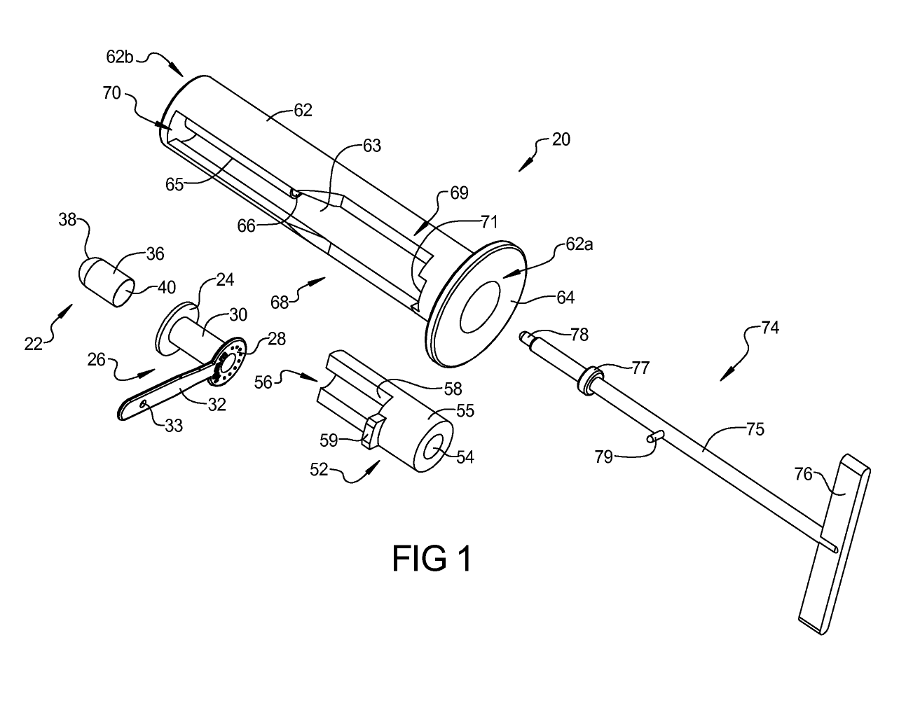

Referring to FIG. 1, a gel cap loading tool 20, according to the present disclosure, is shown. Loading tool 20 is operable to place a gel cap 22 over a component of a medical device having a radial profile that is to be reduced in dimension and inserted into gel cap 22, such as an esophageal flange 24 of a voice prosthesis 26, by way of non-limiting example. Voice prosthesis 26 includes esophageal flange 24 and a tracheal flange 28 with a generally cylindrical hollow body 30 extending there between. Body 30 is open on both flanges 24, 28. A one-way valve (not shown) is disposed within body 30. A strap 32 extends outwardly from tracheal flange 28. Strap 32 has an opening 33 adjacent its end. The depiction of voice prosthesis 26 is representative of typical voice prosthesis. It should be appreciated, however, that voice prosthesis 26 can have a different configuration than that shown and still be utilized with the loading tool 20 according to the present disclosure.

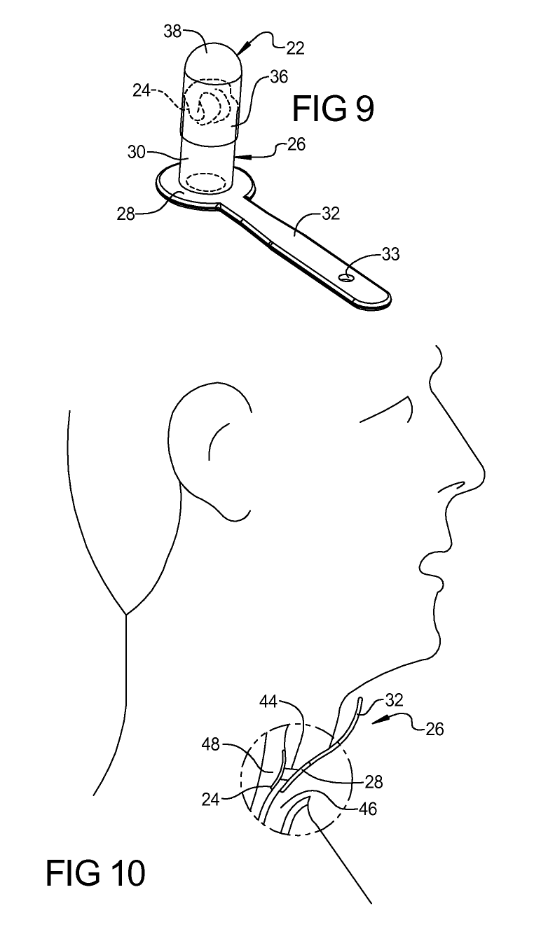

Gel cap 22 includes a generally cylindrical body portion 36 and a hemispherical end portion 38. Body portion 36 and end portion 38 define an interior cavity 40 within which esophageal flange 24 and a portion of body 30 of voice prosthesis 26 can be inserted, as shown in FIG. 9. Gel cap 22 is dissolvable and facilitates the insertion of voice prosthesis 26 into a tracheoesophageal puncture 44 that extends between a trachea 46 and esophagus 48 of a user, as shown in FIG. 10. In FIG. 10, gel cap 22 has been dissolved such that esophageal flange 24 is fully expanded within esophagus 48. Esophageal and tracheal flanges 24, 28 thereby retain voice prosthesis 26 in the tracheoesophageal puncture 44, as known in the art. Suitable gel caps 22 include those available from Qualicaps, Inc of Whitsett, N.C., such as Part Nos. NF 0101 (16 Fr.) and NF 0100 (20 Fr.).

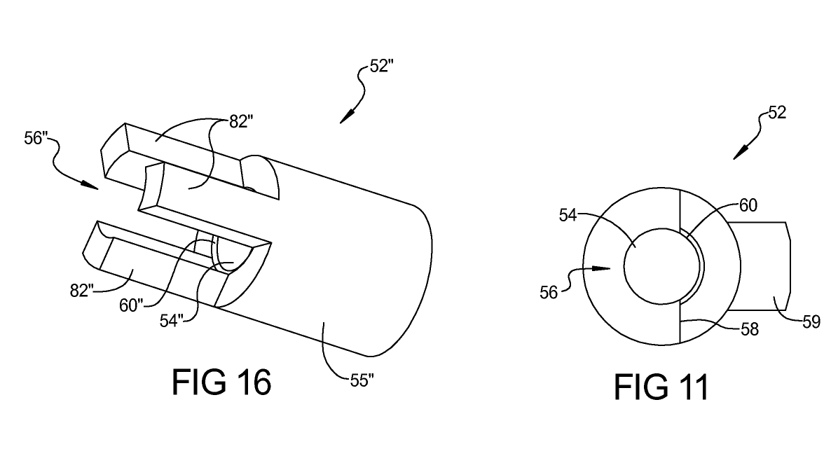

Referring to FIGS. 1 and 11, loading tool 20 includes a generally cylindrical sleeve 52 with a cylindrical opening/cavity 54 extending longitudinally therethrough. A portion of a cylindrical periphery 55 of sleeve 52 is missing to provide a loading opening 56. As shown in FIG. 11, loading opening 56 extends less than 180 degrees about the cylindrical periphery 55 and provides a substantially flat surface 58 which is off-center of a longitudinal axis of sleeve 52. Loading opening 56 facilitates the insertion of voice prosthesis 26 into sleeve 52, as described below. A tab 59 extends radially outwardly from an end of loading opening 56. The access to opening 54 through loading opening 56 can include a chamfer 60 to facilitate the insertion of voice prosthesis 26 into opening 54. When inserted into opening 54, the diameter of opening 54 dictates the compression/collapsing of esophageal flange 24 therein. The diameter of opening 54 is chosen so that esophageal flange 24 collapses a sufficient amount to fit within interior 40 of gel cap 22 without causing undue stress on esophageal flange 24 and gel cap 22 during the loading process.

Loading tool 20 includes an elongated generally cylindrical barrel 62 with opposite ends 62a, 62b and with a cylindrical interior cavity 63 that extends longitudinally therebetween. End 62a is open to cavity 63 and includes a flange 64 that extends radially outwardly therefrom. Barrel 62 includes a generally cylindrical stop 65 that extends longitudinally from closed end 62b toward open end 62a. The outer diameter of stop 65 is smaller than the inner diameter of cavity 63 such that stop 65 is completely spaced apart from the inner surface of cavity 63. Stop 65 can be coaxial with barrel 62 and cavity 63. Stop 65 includes a concave recess 66 on the free end thereof. Stop 65 is fixed in position within barrel 62. The outer diameter of stop 65 is smaller than the inner diameter of opening 54 in sleeve 52 to allow sleeve 52 to slide over stop 65.

Barrel 62 includes a longitudinally extending access opening 68 that extends from the closed end 62b of barrel 62 toward flange 64. Opening 68 includes a wide portion 69 and a narrow portion 70. Wide portion 69 is dimensioned to allow sleeve 52 to be inserted into cavity 63 of barrel 62. Wide portion 69 includes a recess 71 that is configured to receive tab 59 of sleeve 52. Narrow portion 70 is dimensioned to allow tab 59 to slide therethrough when sleeve 52 is slid within cavity 63.

An implement, such as an inserter stick 74, can be used to insert voice prosthesis 26 within tracheoesophageal puncture 44. Inserter stick 74 includes a stem 75 with a handle 76 at one end thereof. There is a radial collar 77 on stem 75 adjacent the opposite end. Collar 77 limits the distance inserter stick 74 can be inserted into voice prosthesis 26. A tip 78 on the end of stem 75 can be tapered to facilitate insertion of stem 75 into voice prosthesis 26 through the opening in tracheal flange 28. A peg 79 extends outwardly from stem 75 between collar 77 and handle 76. Peg 79 is configured to receive strap 32 with peg 79 extending through opening 33. Peg 79 is positioned so that strap 32 is stretched to engage with peg 79. The stretching of strap 32 helps keep voice prosthesis 26 on inserter stick 74 during handling.

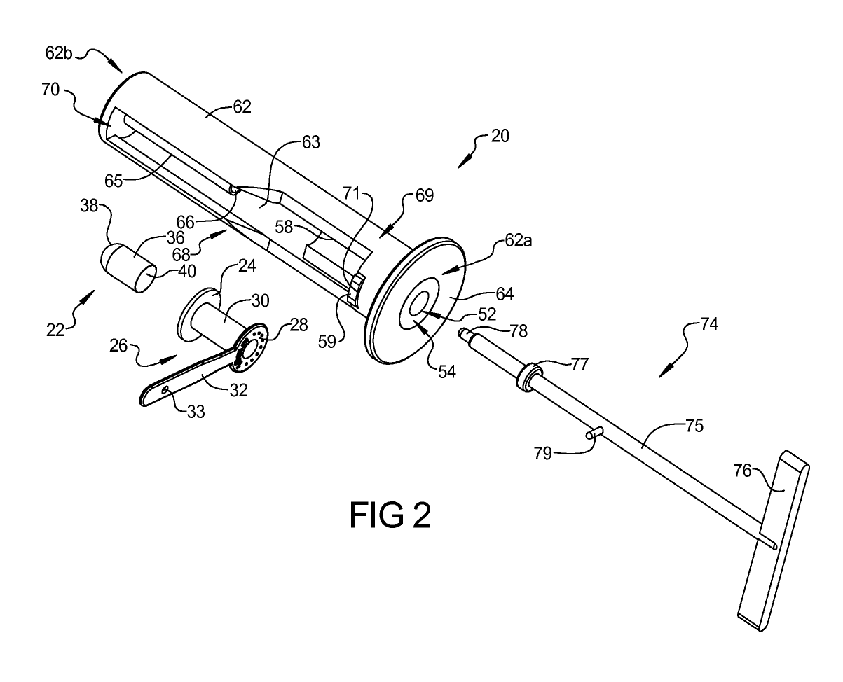

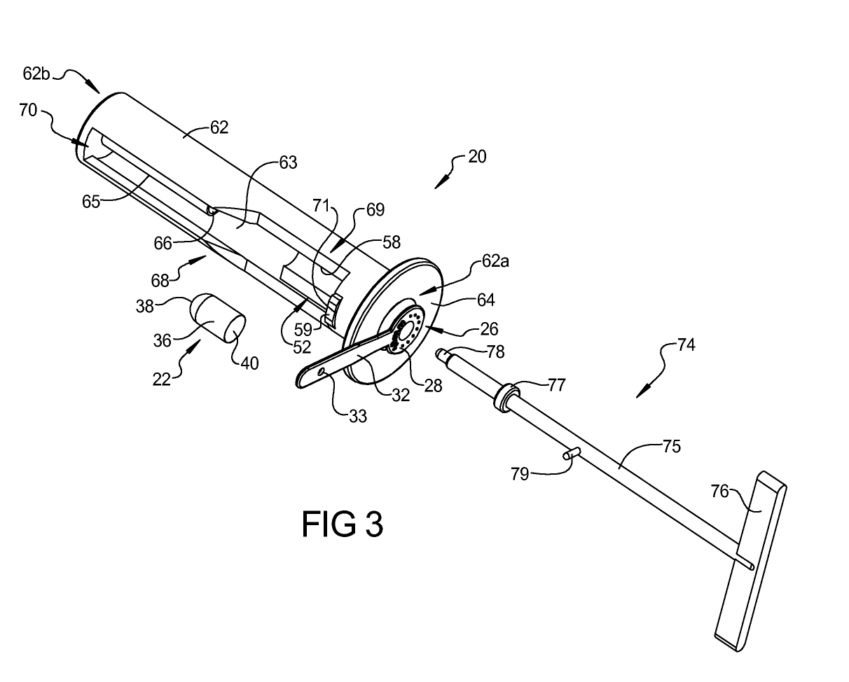

Referring now to FIGS. 2-8, the operation of loading tool 20 to insert esophageal flange 24 of voice prosthesis 26 within gel cap 22 is shown. Initially, as shown in FIG. 2, sleeve 52 is disposed within cavity 63 of barrel 62. Sleeve 52 is positioned adjacent flange 64 with tab 59 engaged in recess 71. Engagement of tab 59 with recess 71 inhibits rotation of sleeve 52 within barrel 62. Next, voice prosthesis 26 is inserted into opening 54 of sleeve 52 by threading strap 32 through loading opening 56 and out opening 54 and end 62a of barrel 62. Strap is threaded so that it extends beyond flange 64 of barrel 62. Strap 32 can then be pulled while the remaining portions of voice prosthesis 26 are inserted into opening 54 through loading opening 56. During the loading process, the flanges 28, 24 will be compressed and collapsed within opening 54, thereby reducing their radial dimensions. When completely seated within sleeve 52, tracheal flange 28 may extend out of opening 54 along the end of sleeve 52 while esophageal flange 24 remains compressed such that its radial dimension is reduced, as shown in FIG. 3. In some embodiments, tracheal flange 28 may remain compressed in opening 54 with its radial dimension reduced along with esophageal flange 24.

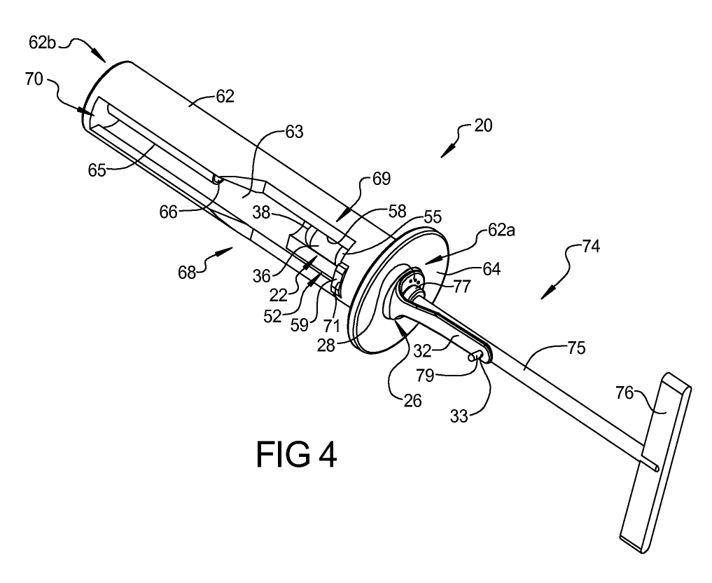

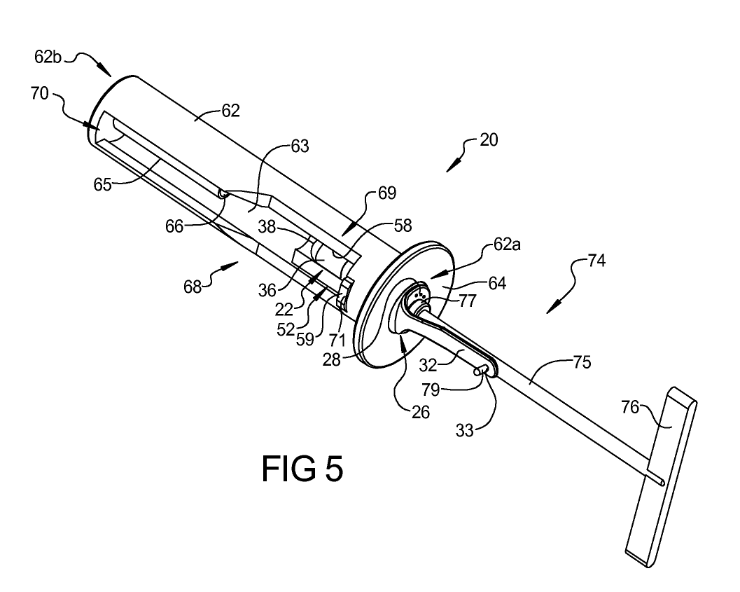

The next step is to load gel cap 22 into sleeve 52. Gel cap 22 is inserted into loading opening 56 of sleeve 52. Because loading opening 56 extends less than 180 degrees around the cylindrical periphery 55, gel cap 22 can be snapped into loading opening 56, as shown in FIG. 4. Opening 54 of sleeve 52 axially aligns voice prosthesis 26 and gel cap 22. Next, as shown in FIG. 5, tip 78 and stem 75 on inserter stick 74 are inserted into voice prosthesis 26 through the opening in tracheal flange 28. Strap 32 is then stretched and secured to inserter stick 74 by engaging peg 79 with opening 33.

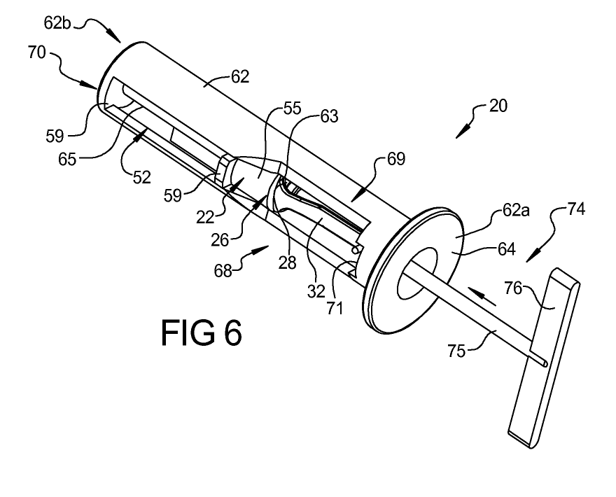

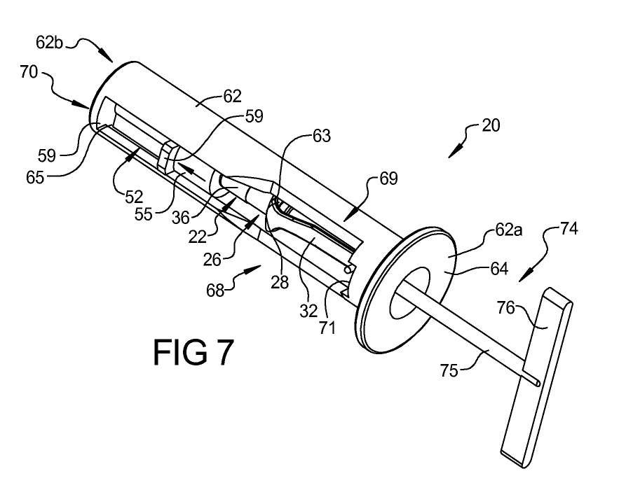

Next, as shown in FIG. 6, sleeve 52 and inserter stick 74 are moved axially relative to barrel 62 so that sleeve 52 approaches stop 65. Flange 64 and handle 76 can be moved toward one another like a syringe to facilitate the movement of inserter stick 74 and sleeve 52 relative to barrel 62. The movement of inserter stick 74 and sleeve 52 relative to barrel 62 also causes voice prosthesis 26 to move with the movement of sleeve 52 and inserter stick 74. The movement eventually causes end 38 of gel cap 22 to engage with concave recess 66 of stop 65. Stop 65 prevents additional movement of gel cap 22 relative to barrel 62. Continued movement of sleeve 52, inserter stick 74, and voice prosthesis 26 causes esophageal flange 24 to enter into interior 40 of gel cap 22. The insertion of esophageal flange 24 into interior 40 can cause some resistance to be felt by the user. Once esophageal flange 24 is completely disposed within gel cap 22, continued movement of inserter stick 74 and voice prosthesis 26 relative to barrel 62 stops. At this time, the user can then continue to move sleeve 52 relative to barrel 62 by pushing tab 59 toward closed end 62b of barrel 62. Sleeve 52 then moves relative to voice prosthesis 26 and gel cap 22 such that sleeve 52 disengages from voice prosthesis 26 and gel cap 22, as shown in FIG. 7.

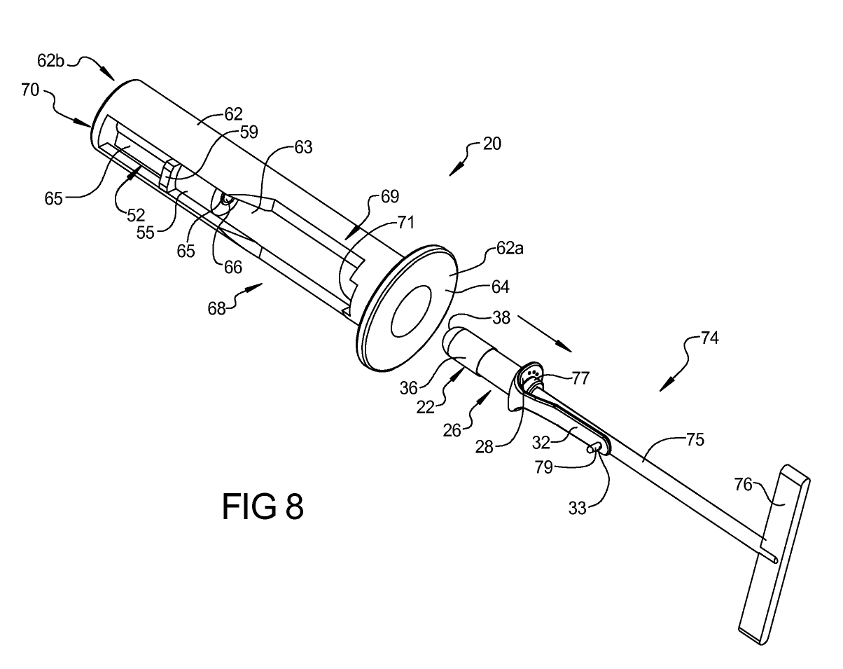

Next, inserter stick 74 is withdrawn from barrel 62. The engagement of strap 32 with peg 79 causes voice prosthesis 26 and gel cap 22 to be removed from barrel 62 along with inserter stick 74, as shown in FIG. 8. The voice prosthesis 26 can then be inserted into the tracheoesophageal puncture 44 and, once inserted, released from inserter stick 74 by disengaging strap 32 from peg 79. Inserter stick 74 can then be removed from voice prosthesis 26 and voice prosthesis 26 will remain within tracheoesophageal puncture 44. The user can then drink a fluid to dissolve gel cap 22, thereby releasing esophageal flange 24 and securing voice prosthesis 26 within tracheoesophageal puncture 44, as shown in FIG. 10.

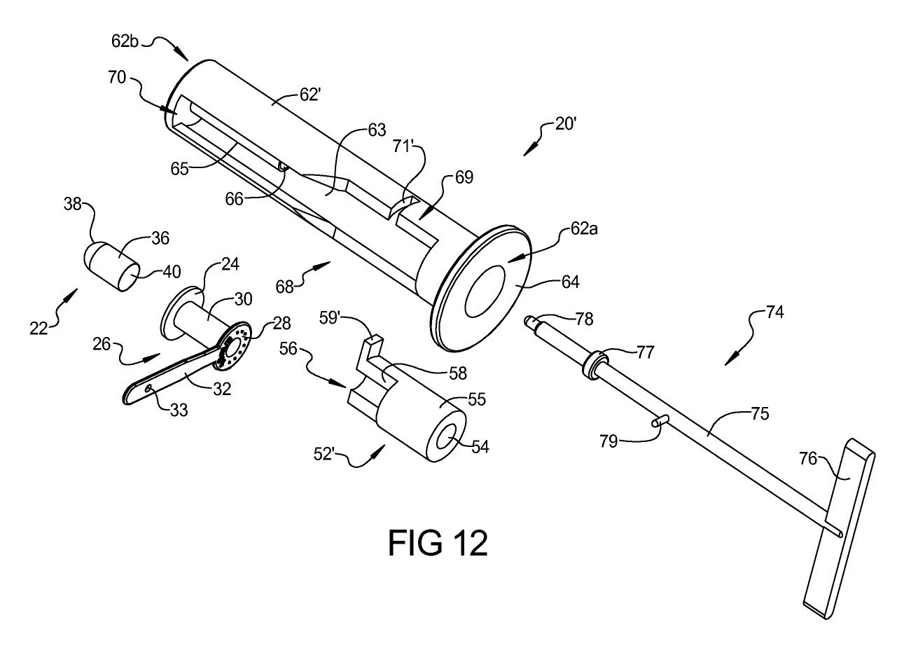

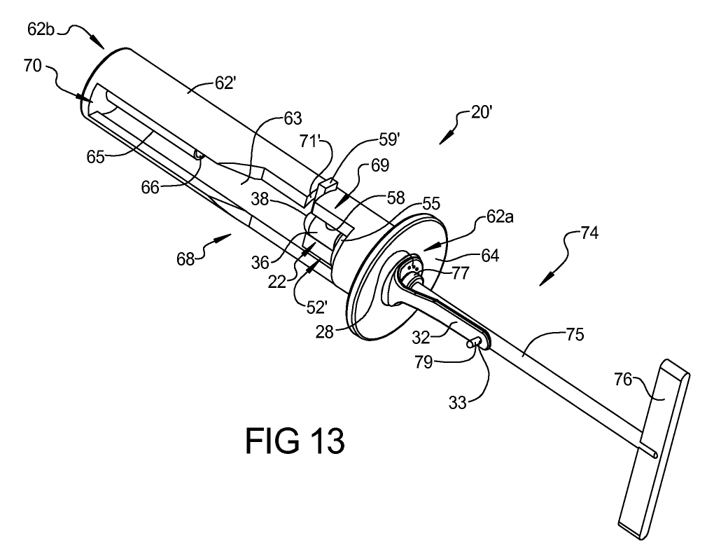

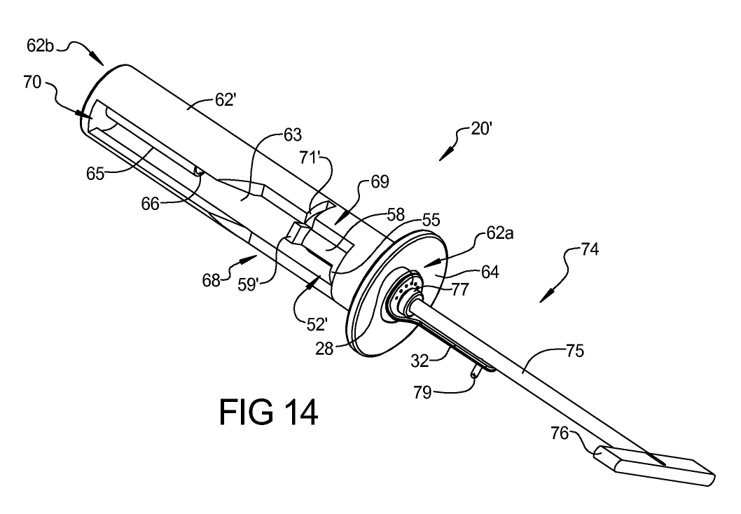

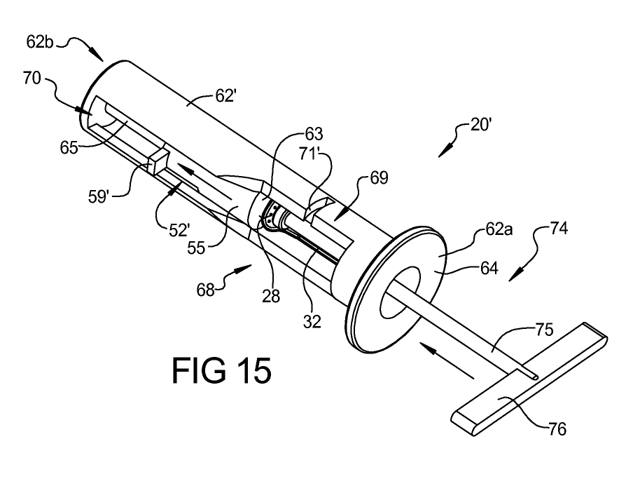

Referring now to FIGS. 12-15, an alternate configuration of a loading tool 20′ is shown. In loading tool 20′, tab 59′ of sleeve 52′ extends radially outwardly from an end of sleeve 52′ adjacent loading opening 56. Additionally, recess 71′ of barrel 62′ extends from a side of wide portion 69 and is configured to receive tab 59′ of sleeve 52′. Specifically, as shown in FIG. 13, when sleeve 52′ is inserted in barrel 62′, sleeve 52′ is rotated so that tab 59′ is engaged with recess 71′. Engagement of tab 59′ with recess 71′ inhibits longitudinal movement of sleeve 52′ within barrel 62′. When using loading tool 20′, sleeve 52′, after receiving voice prosthesis 26 and gel cap 22, is rotated within barrel 62′ such that tab 59′ is disengaged from recess 71′, as shown in FIG. 14. With tab 59′ disengaged from recess 71′, sleeve 52′ and inserter stick 74 are moved axially relative to barrel 62′ so that sleeve 52′ approaches stop 65 and causes esophageal flange 24 to enter interior 40 of gel cap 22, in the same manner described above with reference to FIGS. 1-8.

Referring now to FIG. 16, another alternate configuration of a sleeve 52″ is shown. In sleeve 52″, a plurality of longitudinally extending arms 82″ extend forwardly from cylindrical periphery 55″. Arms 82″ are spaced apart to form a loading opening 56″ that leads to opening 54″. A chamfer 60″ can surround the access to opening 54″ through loading opening 56″. In this embodiment, voice prosthesis 26 is inserted into opening 54″ by squeezing voice prosthesis 26 through the loading openings 56″ formed between arms 82″. Additionally, sleeve 52″ does not include a tab. Rather, in this embodiment, sleeve 52″ can be moved forwardly with movement of inserter stick 74 relative to barrel 62. When gel cap 22 is engaged with concave recess 66 of stop 65, further forward movement of sleeve 52″ can be performed by a user using their fingers to push sleeve 52″ forwardly such that sleeve 52″ disengages from voice prosthesis 26 and gel cap 22.

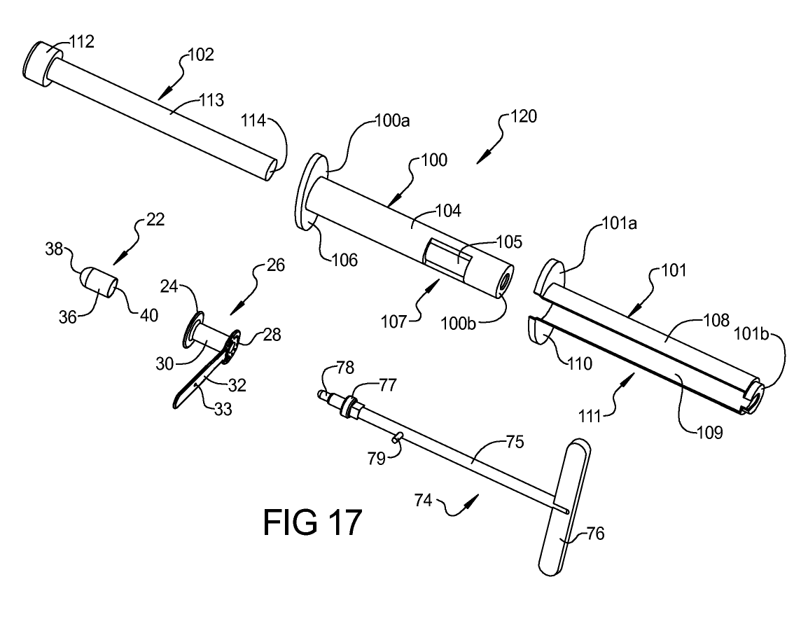

Referring now to FIGS. 17-21, another loading tool 120, according to the present disclosure, is shown. Loading tool 120 is operable to place gel cap 22 over esophageal flange 24 of voice prosthesis 26. Loading tool 120 includes inner and outer barrels 100, 101 and a plunger 102. Inner barrel 100 is generally cylindrical with an exterior peripheral surface 104 and an interior cylindrical cavity 105 that extends longitudinally throughout the entire length of inner barrel 100. Cavity 105 is accessible from both ends 100a, 100b of inner barrel 100. End 100a includes a flange 106 that extends radially outwardly therefrom. Inner barrel 100 includes an access opening 107 in peripheral surface 104. Access opening 107 allows voice prosthesis 26 and gel cap 22 to be inserted into cavity 105, as described below.

Outer barrel 101 is also cylindrical with an exterior peripheral surface 108 and an interior cylindrical cavity 109. Cavity 109 extends longitudinally within outer barrel 101 an entire length of outer barrel 101. Cavity 109 is accessible from both ends 101a, 101b of outer barrel 101. Outer barrel 101 includes a flange 110 that extends radially outwardly from end 101a. Outer barrel 101 includes an access opening 111 in peripheral surface 108 that extends an entire length of outer barrel 101 between ends 101a, 101b. Cavity 109 is dimensioned to allow inner barrel 100 to be disposed therein, as described below.

Plunger 102 includes a head 112 and an elongated cylindrical stem 113 extending therefrom. Stem 113 has an outer diameter dimensioned to fit within cavity 105 of inner barrel 100 through flange end 100a. Head 112 is larger than the opening to cavity 105 in flange end 100a and limits the travel of plunger 102 within inner barrel 100. The free end 114 of plunger 102 can include a concave recess to engage with end 38 of gel cap 22. Plunger 102 is operable to push gel cap 22 over esophageal flange 24 of voice prosthesis 26 within inner barrel 100, as described below.

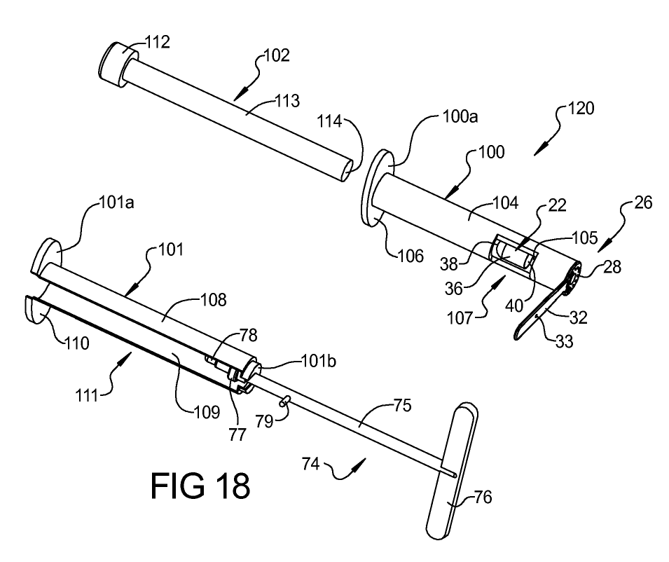

To use loading tool 120, voice prosthesis 26 can be loaded into inner barrel 100 through access opening 107. Specifically, strap 32 can be threaded through access opening 107 and out of end 100b. Strap 32 can be used to pull voice prosthesis 26 into cavity 105. When seated within inner barrel 100, tracheal flange 28 may extend out of inner barrel 100 through end 100b while esophageal flange 24 is collapsed/compressed within cavity 105, as shown in FIG. 18, such that its radial dimension is reduced. In some embodiments, tracheal flange 28 may remain compressed within cavity 105 along with esophageal flange 24. Next, gel cap 22 can be inserted into cavity 105 through access opening 107, also as shown in FIG. 18. Voice prosthesis 26 and gel cap 22 are axially aligned by cavity 105. Inserter stick 74 is inserted into cavity 109 of outer barrel 101 through access opening 111 with collar 77 disposed in cavity 109 inside of end 101b, as shown in FIG. 18.

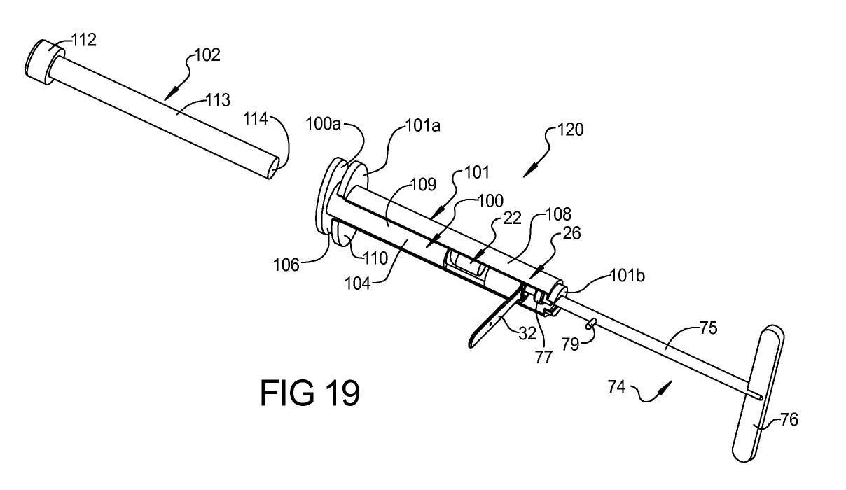

Next, inner barrel 100 is inserted into cavity 109 of outer barrel 101 through the opening in flange end 101a. Strap 32 can extend out of outer barrel 101 through access opening 111, as shown in FIG. 19. Inner barrel 100 is moved (to the right as shown in the figures) within outer barrel 101 to seat stem 75 of inserter stick 74 in voice prosthesis 26 through the opening in tracheal flange 28. Inner and outer barrels 100, 101 can be moved relative to one another in a syringe-like manner through the use of flanges 106, 110.

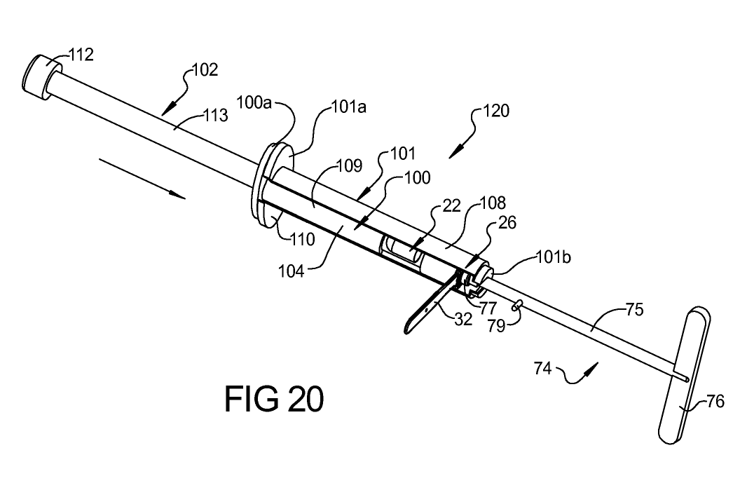

Next, as shown in FIG. 20, stem 113 of plunger 102 is inserted into cavity 105 of inner barrel 100 through the opening in flange end 100a. Loading tool 120 is now ready to be operated to push gel cap 22 over esophageal flange 24. To accomplish this, head 112 of plunger 102 is pushed toward flange end 100a of inner barrel 100. This can be accomplished using a syringe-like motion by holding onto flange 110 of outer barrel 101 while pushing on head 112 of plunger 102.

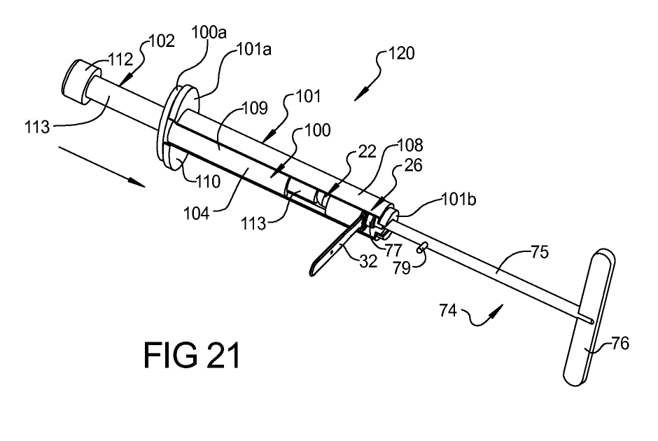

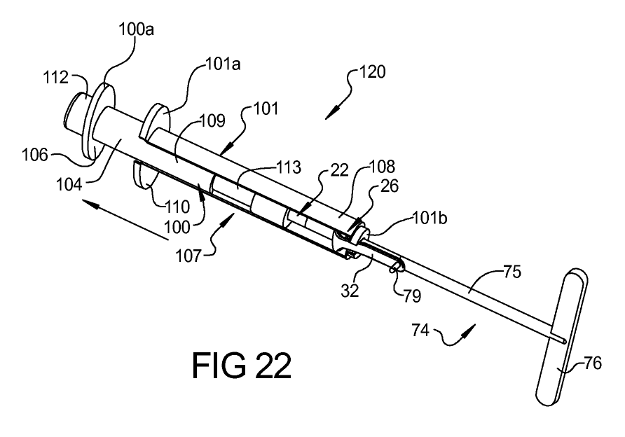

The movement of plunger 102 relative to inner barrel 100 causes stem 113 to push gel cap 22 relative to voice prosthesis 26 and onto esophageal flange 24 and a portion of body 30, as shown in FIG. 21. With gel cap 22 fully loaded on voice prosthesis 26, inner barrel 100 can be moved (to the left as shown in the figures) relative to outer barrel 101 to release voice prosthesis 26 with gel cap 22 thereon from cavity 105 of inner barrel 100 through the opening in end 10b, as shown in FIG. 22.

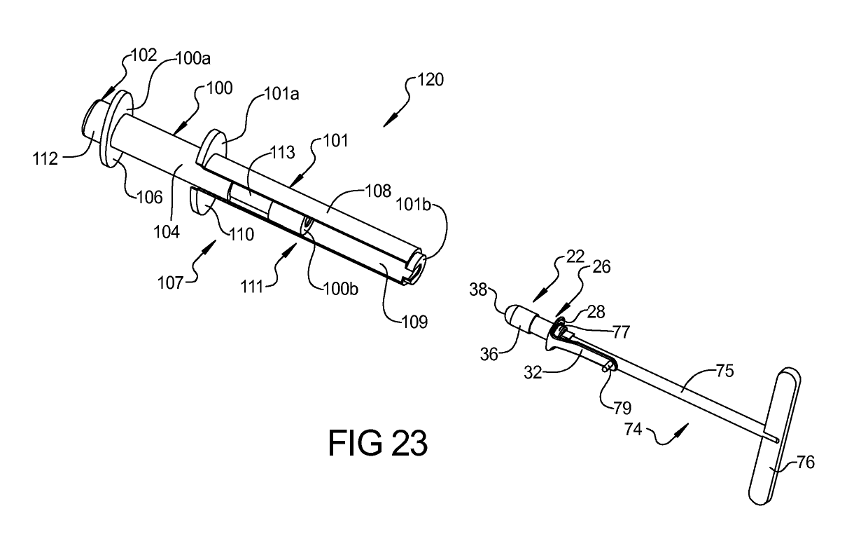

With gel cap 22 and voice prosthesis 26 being removed from cavity 105 of inner barrel 100, voice prosthesis 26 and gel cap 22 can be removed from outer barrel 101 through access opening 111, as shown in FIG. 23. Inserter stick 74 can then be utilized to place voice prosthesis 26 into the tracheoesophageal puncture 44 of a user, as shown in FIG. 10 and as described above. Thus, loading tool 120 can be utilized to position a gel cap 22 over an esophageal flange 24 of a voice prosthesis 26.

The various components of loading tools 20, 20′, 120 can be made from a variety of materials. Exemplary materials include polypropylene and ABS plastic. Furthermore, the various components may be transparent or translucent to facilitate the use of the tools. It should be appreciated, however, that other materials can be used and/or one or more of the components may be opaque.

It should be appreciated that while loading tools 20, 20′, 120 are disclosed as being used with a voice prosthesis 26 which is disclosed as being used in a tracheoesophageal puncture 44, loading tools 20, 20′, 120 can be used for other types of medical devices having a radial dimension that is to be reduced by, insertion into a gel cap 22 or other cap for subsequent insertion into an orifice. By way of non-limiting example, loading tools 20, 20′, 120 can be used on a prosthesis intended to be inserted into an opening in a nasal septum or a puncture through a tympanic membrane.

While loading tools 20, 20′, 120 are shown with reference to specific structures and with specific sequences of loading and operating, it should be appreciated that such structures and loading operations are merely exemplary and that deviations to these can occur. For example, the sequence in which voice prosthesis 26, gel cap 22, and the components of loading tool 20, 20′, 120 are assembled together and operated can deviate. As another example, the loading tool can be provided in a double-barrel configuration to accommodate voice prosthesis of varying sizes. That is, multiple barrels can be utilized with each barrel and the associated components dimensioned for different sizes of voice prosthesis such that a single loading tool can be utilized to load gel caps over voice prosthesis of varying sizes. Additionally, other implements can be used in lieu of inserter stick 74. Thus, the loading tools 20, 20′, 120 and their descriptions are merely exemplary and deviations that depart from these exemplary structures and sequence of events are intended to be included in the scope of the present disclosure.

What is claimed is:

1. A loading tool for loading a capsule on a medical device, the loading tool comprising:
a device section configured to receive a medical device therein;
a capsule section configured to receive a capsule therein; and
an engaging member having an engaging surface thereon that interacts with said capsule section and said device section,
wherein relative movement between said engaging member and said device section and said capsule section causes a component of a medical device in said device section to be reduced in radial dimension and inserted into a capsule in said capsule section.

2. The loading tool of claim 1, wherein said device section and said capsule section are co-axial with one another and said engaging member and said sections move axially relative to one another.

3. The loading tool of claim 2, further comprising:
an elongated barrel having an interior within which said engaging member is disposed; and
a moveable member disposed in said interior of said barrel and moveable therein, said moveable member having an axially extending cavity therein that includes said device section and said capsule section,
wherein movement of said moveable member within said barrel causes said engaging member to enter said cavity of said moveable member and is operable to insert a component of a medical device into a capsule.

4. The loading tool of claim 3, wherein said barrel and said moveable member are substantially cylindrical in shape.

5. The loading tool of claim 3, wherein said cavity of said moveable member is open on both ends and one component of said medical device extends out of said cavity and another component of said medical device is collapsed within said cavity when said medical device is loaded into said cavity and ready to be inserted into said capsule.

6. The loading tool of claim 3, wherein one end of said barrel includes an access opening communicating with said interior and is configured to receive an implement therethrough for attaching to said medical device.

7. The loading tool of claim 3, wherein one end of said barrel includes an access opening communicating with said interior, one end of said moveable member can be disposed in said access opening, and one component of said medical device extends out of said cavity and said barrel through said access opening and another component of said medical device is collapsed within said cavity when said medical device is loaded into said cavity and ready to be partially inserted into said capsule.

8. The loading tool of claim 3, wherein said barrel includes an access opening that extends axially along a peripheral surface of said barrel and communicates with said interior, said access opening configured to allow a medical device and a capsule to be loaded into said cavity of said moveable member through said access opening.

9. The loading tool of claim 3, wherein said engaging member is fixed relative to said barrel in said interior.

10. The loading tool of claim 2, further comprising:
a first elongated barrel having a first interior;
a second elongated barrel having a second interior, said second barrel being disposed in said first interior of said first barrel and moveable therein, said second interior including said device section and said capsule section,
wherein said engaging member is insertable into said second interior and moveable therein to insert a component of a medical device into a capsule.

11. The loading tool of claim 10, wherein said first and second barrels are substantially cylindrical barrels with openings on each end that communicate with said first and second interiors, respectively, said first barrel has an axially extending access opening along a periphery thereof communicating with said first interior, said second barrel includes an axially extending loading opening along a periphery thereof communicating with said second interior and configured to allow said medical device and said capsule to be loaded into said second interior, and said engaging member is inserted into said second barrel through a first one of said end openings.

12. The loading tool of claim 11, wherein said medical device is a voice prosthesis, one flange of said voice prosthesis extends out of said second interior through a second one of said second barrel end openings and another flange of said voice prosthesis is collapsed within said second interior when said voice prosthesis is loaded into said second interior and ready to be inserted into said capsule.

13. The loading tool of claim 12, wherein said one flange of said voice prosthesis extends out of said second interior and into said first interior when said voice prosthesis is loaded into said second interior and ready to be inserted into said capsule.

14. A loading tool for loading a capsule on a medical device, the loading tool comprising:
a first member having an elongated cavity configured to receive a medical device and a capsule therein; and
a second member configured to enter said cavity and engage with at least one of a medical device and a capsule disposed in said cavity,
wherein relative movement between said first and second members causes said second member to enter said cavity and a component of a medical device disposed in said cavity to be radially reduced in dimension and inserted into a capsule disposed in said cavity.

15. The loading tool of claim 14, wherein said cavity axially aligns a medical device and a capsule disposed therein.

16. The loading tool of claim 14, further comprising an elongated third member having an outer periphery and an interior and wherein said first member is moveably disposed in said interior of said third member.

17. The loading tool of claim 16, wherein said third member has an end with an opening therein communicating with said interior and said opening is configured to receive an implement therein which can engage with a medical device disposed in said cavity.

18. The loading tool of claim 17, wherein said third member has an access opening extending axially along said periphery and a medical device and a capsule are loaded into said first member through said access opening and are removed from said cavity through said opening in said end of said third member.

19. The loading tool of claim 16, wherein said second member is disposed in said interior and is stationary relative to said third member.

20. The loading tool of claim 19, wherein movement of said first member within said third member causes a component of a medical device disposed in said cavity to be inserted into a capsule disposed in said cavity.

21. The loading tool of claim 16, wherein said second member is moveably disposed in said interior and in said cavity and movement of said second member within said interior and said cavity causes a component of a medical device disposed in said cavity to be inserted into a capsule disposed in said cavity.

22. The loading tool of claim 14, wherein a different component of a medical device extends out of said cavity when loaded therein.

23. A method of inserting a component of a medical device into a capsule, the method comprising:
inserting a medical device into a cavity in a first member;
inserting a capsule into said cavity;
moving one of said medical device and said capsule relative to one another in said cavity such that a component of said medical device is inserted into said capsule; and
removing said medical device with said capsule thereon from said cavity.

24. The method of claim 23, wherein inserting said medical device into said cavity includes deforming said component in said cavity to a radial dimension less than a non-deformed radial dimension of said component.

25. The method of claim 23, wherein inserting said medical device and said capsule includes axially aligning said medical device and said capsule with a peripheral wall of said cavity.

26. The method of claim 23, wherein moving one of said medical device and said capsule includes engaging one of said medical device and said capsule with an engaging member and moving said first member and said engaging member relative to one another.

27. The method of claim 26, wherein said first member is disposed in an interior of a second member, said engaging member is stationarily disposed in said interior of said second member, and moving one of said medical device and said capsule includes moving said first member axially within said interior of said second member.

28. The method of claim 26, wherein moving said first member and said engaging member includes moving said engaging member axially within said cavity relative to said first member.

29. The method of claim 26, wherein said first member is disposed in an interior of a second member, said engaging member is disposed in said cavity and in said interior, and moving one of said medical device and said capsule includes moving said engaging member axially within said interior of said second member and axially within said cavity of said first member.

30. The method of claim 23, further comprising inserting an end of an implement into said medical device through an opening in a component of said medical device opposite said capsule while said medical device is disposed in said cavity.

31. The method of claim 30, wherein removing said medical device and said capsule from said cavity includes removing said implement from the tool with said medical device and said capsule disposed thereon.

32. The method of claim 23, wherein inserting said medical device and said capsule include inserting said medical device and said capsule into said cavity through a first opening in said first member and removing said medical device with said capsule thereon includes removing said medical device with said capsule thereon from said cavity through a second opening in said first member distinct from said first opening.

33. The method of claim 23, wherein said medical device is a voice prosthesis and said component is a flange of said voice prosthesis.
